(19) United States
(12) Patent Application Publication
(10) Pub. No.: US 2016/0004843 A1
(43) Pub. Date: Jan. 7, 2016

(54) INSPECTION DATA DISPLAY CONTROL APPARATUS, METHOD, AND RECORDING MEDIUM

(71) Applicant: FUJIFILM Corporation, Tokyo (JP)

(72) Inventor: TAKASHI HOSHINO, Kanagawa (JP)

(21) Appl. No.: 14/850,926

(22) Filed: Sep. 10, 2015

Related U.S. Application Data
(63) Continuation of application No. PCT/JP2014/001600, filed on Mar. 20, 2014.

(30) Foreign Application Priority Data
Mar. 29, 2013 (JP) 2013-071272

(51) Int. Cl. G06F 19/00; A61B 5/00

(52) U.S. Cl. CPC G06F 19/3487; A61B 5/7425; A61B 5/0022

(57) ABSTRACT

An inspection data display control apparatus for displaying inspection data of a plurality of inspection items obtained in time series includes a display period specification receiving unit that receives a specification of a display period of inspection data to be displayed, an inspection data obtaining unit that, when the specification of the display period is received, identifies an inspection item whose inspection data are present in the display period from the plurality of inspection items and obtains only the inspection data of the identified inspection item, as display target inspection data present in the display period, and a display control unit that displays only the inspection data of the inspection item present in the display period.

### CROSS-REFERENCE TO RELATED APPLICATIONS

This application is a Continuation of PCT International Application No. PCT/JP2014/001600 filed on Mar. 20, 2014, which claims priority under 35 U.S.C. §119(a) to Japanese Patent Application No. 2013-071272 filed on Mar. 29, 2013. Each of the above applications is hereby expressly incorporated by reference, in its entirety, into the present application.

### BACKGROUND

The present disclosure relates to an inspection data display control apparatus, method, and program for displaying inspection data of a plurality of inspection items obtained in time series.

Recently, when a medical examination is performed on a patient by a doctor, it has been practiced to display a plurality of inspection results performed on the patient as reference information for determining the disease name and treatment policy.

A wide variety of inspections is performed on a patient and if a patient is suspected to have a specific disease, diagnosis or treatment is performed with reference to each inspection result of a plurality of inspections considered as important for the diagnosis of the specific disease in actual medical sites.

But, there may be a case where inspection items amount to a large number and if the patient is suffering from a plurality of diseases, the number of inspection items is further increased. Therefore, if all inspection results of the many inspection items are displayed on top of each other on the same coordinate axis as graphs, it is very difficult to clearly see the graphs as they overlap with each other, whereby even if there is an abnormal sign in the inspection data, the sign may possibly be overlooked.

Hence, for example, Japanese Unexamined Patent Publication No. 2001-133293 and Japanese Unexamined Patent Publication No. 9 (1997)-185651 disclose a method for displaying inspection data by limiting the display period of graphs of inspection data by receiving a specification of a display period of interest and displaying graphs of inspection data within the received display period.

### SUMMARY

But, even if the graphs are displayed by narrowing down only the display period, as in Japanese Unexamined Patent Publication No. 2001-133293 and Japanese Unexamined Patent Publication No. 9 (1997)-185651, graph lines of many inspection items having no inspection data in the display period are displayed, whereby it is difficult to clearly see the graphs after all.

In view of the circumstances described above, the present disclosure provides an inspection data display control apparatus, method, and program capable of displaying inspection data of a plurality of inspection items in an easy-to-view state.

An inspection data display control apparatus of the present disclosure is an inspection data display control apparatus for displaying inspection data of a plurality of inspection items obtained in time series, the apparatus including a display period specification receiving unit that receives a specification of a display period of inspection data to be displayed, an inspection data obtaining unit that, when the specification of the display period is received, identifies an inspection item whose inspection data are present in the display period from the plurality of inspection items and obtains only the inspection data of the identified inspection item, as display target inspection data present in the display period, and a display control unit that displays only the inspection data of the inspection item present in the display period.

In the inspection data display control apparatus of the present disclosure described above, the display period specification receiving unit may receive a specification of a display period changed from the display period, when the specification of the display period before the change is received, the inspection data obtaining unit may identify an inspection item present in display periods preceding and following the display period before the change, and obtain inspection data of the identified inspection item in the display periods preceding and following the display period before the change in advance before the change in the display period, and when the change in the display period is received, the display control unit may display inspection data present in the display period after the change using the inspection data obtained in advance.

Further, when the specification of the display period is received, the inspection data obtaining unit may identify an inspection item present in display periods preceding and following the received display period, and obtain inspection data of the identified inspection item in the display periods preceding and following the received display period in advance, and when displaying the inspection data of the inspection item present in the display period, the display control unit may, if inspection data of the same inspection item as that present in the display period is present in the display periods preceding and following the display period, display the inspection data present in the display period by connecting the inspection data present in the display period with the inspection data of the same inspection item present in the display periods preceding and following the display period.

Still further, when displaying the inspection data present in the display period before the change, the display control unit may identify an inspection item present in the display periods preceding and following the display period before the change, and automatically change a numerical value range of an axis representing numerical values such that maximum and minimum values of each identified inspection item are all displayed in a display area.

Further, when displaying the inspection data present in the display period after the change, the display control unit may automatically shift a numerical value range of an axis representing numerical values such that both maximum and minimum values of the inspection data of the inspection item present in the display period after the change are included in a display area.

Still further, the display control unit may shift the numerical value range by scrolling the axis representing numerical values.

Further, the display control unit may automatically shift the numerical value range of the axis representing numerical values each time a period of a minimum unit of change of the display period is changed.

Still further, the display control unit may display a scroll bar for instructing a change in the display period.

Further, the inspection data obtaining unit may obtain the inspection data via a network.

An inspection data display control method of the present disclosure is an inspection data display control method for displaying inspection data of a plurality of inspection items obtained in time series, the method including the steps of receiving a specification of a display period of inspection data to be displayed, identifying, when the specification of the display period is received, an inspection item whose inspection data are present in the display period from the plurality of inspection items and obtaining only the inspection data of the identified inspection item, as display target inspection data present in the display period, and displaying only the inspection data of the inspection item present in the display period.

An inspection data display control program of the present disclosure is an inspection data display control method for displaying inspection data of a plurality of inspection items obtained in time series, the program causing a computer to function as a display period specification receiving unit that receives a specification of a display period of inspection data to be displayed, an inspection data obtaining unit that, when the specification of the display period is received, identifies an inspection item whose inspection data are present in the display period from the plurality of inspection items and obtains only the inspection data of the identified inspection item, as display target inspection data present in the display period, and a display control unit that displays only the inspection data of the inspection item present in the display period.

According to the inspection data display control apparatus, method, and program of the present disclosure, when displaying inspection data of a plurality of inspection items obtained in time series, a specification of a display period of inspection data to be displayed is received, when the specification of the display period is received, an inspection item whose inspection data are present in the display period is identified from the plurality of inspection items and only the inspection data of the identified inspection item are obtained, as display target inspection data present in the display period, and only the inspection data of the inspection item present in the display period are displayed. This may limit the inspection items of inspection data to be displayed to those present in the specified display period and, therefore, graph lines of inspection items having no inspection data in the display period are not displayed, whereby the graphs may be displayed in a more easy-to-view manner.

### BRIEF DESCRIPTION OF THE DRAWINGS

FIG. 1 is a block diagram of a medical care information display system that uses an embodiment of the inspection data display control apparatus of the present disclosure, illustrating a schematic configuration thereof.

FIG. 2 shows an example of a medical care information display screen.

FIG. 3 is a flowchart for explaining an operation of a medical care information display system that uses a first embodiment of the inspection data display control apparatus of the present disclosure.

FIG. 4 is a table showing an example of inspection data.

FIG. 5 is a drawing for explaining a graph display of inspection data in the medical care information display system of the first embodiment of the present disclosure.

FIG. 6 is a flowchart for explaining an operation of a medical care information display system that uses a second embodiment of the inspection data display control apparatus of the present disclosure.

FIG. 7 is a drawing for explaining a graph display of inspection data in the medical care information display system of the second embodiment of the present disclosure.

FIG. 8 is a flowchart for explaining an operation of a medical care information display system that uses a third embodiment of the inspection data display control apparatus of the present disclosure.

FIG. 9 is a table showing an example of inspection data.

FIG. 10 is a drawing for explaining a graph display of inspection data in the medical care information display system of the third embodiment of the present disclosure.

FIG. 11 shows an example in which inspection data of each inspection item are graph displayed without considering maximum and minimum values of the inspection data.

FIG. 12 is a flowchart for explaining an operation of a medical care information display system that uses a fourth embodiment of the inspection data display control apparatus of the present disclosure.

FIG. 13 is a drawing for explaining a graph display of inspection data in the medical care information display system of the fourth embodiment of the present disclosure.

FIG. 14 is a drawing for explaining a graph display of inspection data in the medical care information display system of the fourth embodiment of the present disclosure.

### DESCRIPTION OF EMBODIMENTS

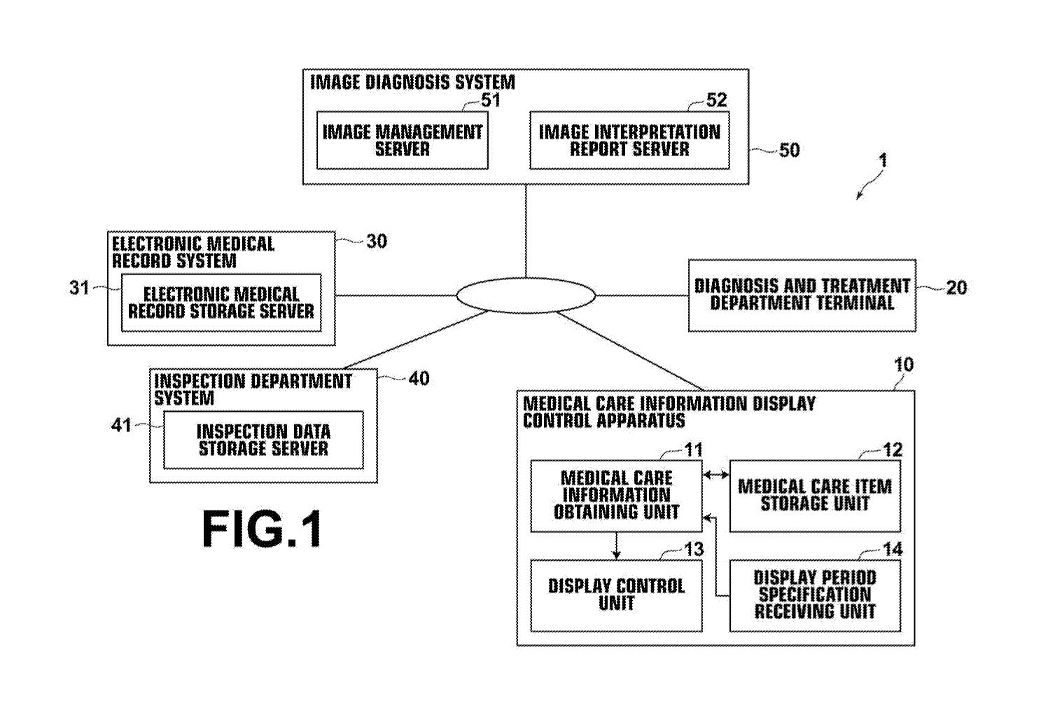

Hereinafter, a medical care information display system that uses a first embodiment of the inspection data display control apparatus, method, and program of the present disclosure will be described in detail with reference to the accompanying drawings. FIG. 1 is a block diagram of a medical care information display system 1 of the present embodiment, illustrating the schematic configuration thereof.

As illustrated in FIG. 1, the medical care information display system 1 of the present embodiment includes a medical care information display control apparatus 10, a diagnosis and treatment department terminal 20, an electronic medical record system 30, an inspection department system 40, and an image diagnosis system 50.

The medical care information display control apparatus 10, the diagnosis and treatment department terminal 20, and each system 30 to 50 in the medical care information display system 1 are connected via a network, such as a wired or wireless LAN, or an Internet circuit.

The medical care information display control apparatus 10 will be described first. Note that the medical care information display control apparatus includes an embodiment of the inspection data display control apparatus of the present disclosure.

The medical care information display control apparatus 10 receives identification information of a patient inputted at the diagnosis and treatment department terminal 20, collects medical care information of the patient from the electronic medical record server 30, the inspection department system 40, and the image diagnosis system 50, generates a medical care information display screen by integrating the collected information, and displays the screen on the diagnosis and treatment department terminal 20.

More specifically, the medical care information display control apparatus 10 includes a medical care information obtaining unit 11 (corresponding to the inspection data obtaining unit), a medical care information storage unit 12, a display control unit 13, and a display period specification receiving unit 14.

The medical care information display control apparatus 10 is a computer on which a medical care information display control program that includes an embodiment of the inspection data display control program of the present disclosure is installed. Execution of the medical care information display control program by the central processing unit (CPU) causes the medical care information obtaining unit 11, the display control unit 13, and the display period specification receiving unit 14 to function. The medical care information display control program described above may be a medical care information display control program recorded on a recording medium, such as a CD-ROM, or down-loaded from a server or the like via the Internet.

The medical care information obtaining unit 11 obtains, based on the identification information of a patient inputted at the diagnosis and treatment department terminal 20, medical care information associated with the identification information of the patient from each server of the system described above via the network. The medical care information includes basic information, such as the name, age, and gender of a patient, medication and drip infusion information, various types of inspection data, such as a sample test, a vital, and the like, and image diagnosis information.

The medical care information obtaining unit 11 temporarily stores the medical care data collected from each server based on the patient identification information in the medical care information storage unit 12. The medical care information storage unit 12 is composed of a storage device, such as a hard disk.

The display control unit 13 generates a medical care information display screen based on the medical care data, including inspection data outputted from the medical care information obtaining unit 11 and outputs the medical care information display screen to the diagnosis and treatment department terminal 20 for display.

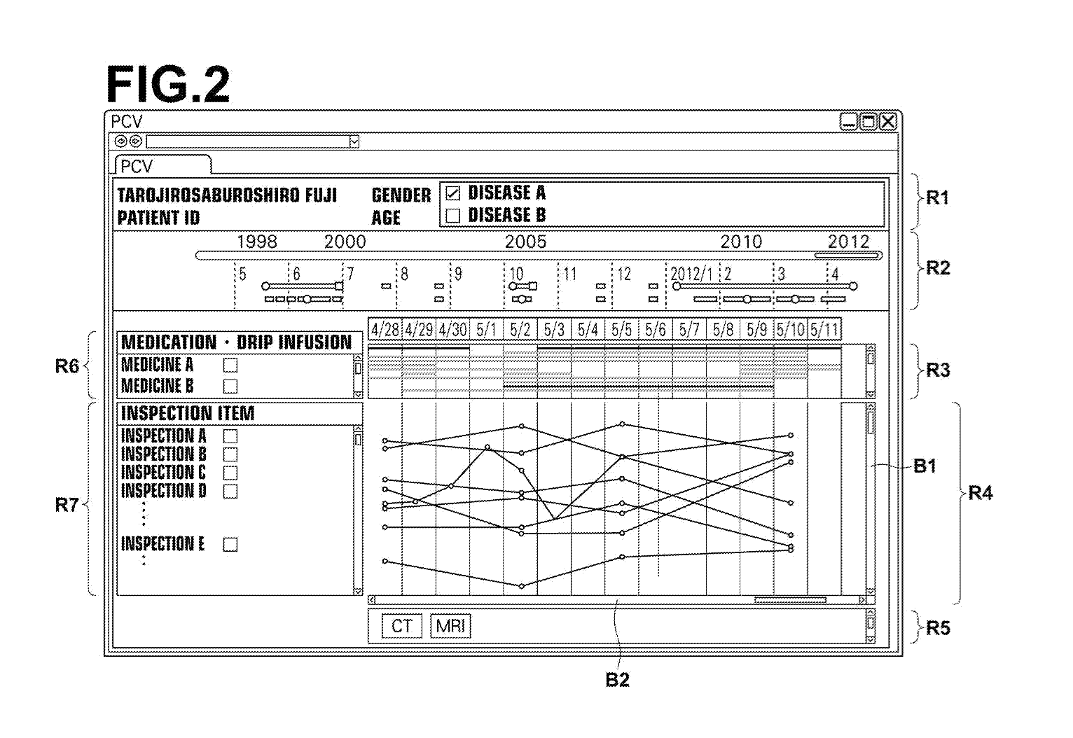

A medical care information display screen to be generated in the display control unit 13 will now be described. FIG. 2 shows an example of a medical care information display screen.

As shown in FIG. 2, the medical care information display screen includes a basic information display section R1, an inspection period display section R2, a medication period display section R3, an inspection data display section R4, an image examination information section R5, a medication list display section R6, and an inspection item list display section R7.

The basic information display section R1 is a section in which basic information of a patient is displayed, such as identification information of a patient (patient ID), name, age, gender, a history of disorders, and the like. For the history of disorders, a checkbox is displayed for each disease, as illustrated in FIG. 2. Then, a disease of interest is specified by checking the checkbox, and inspection data and image examination information of the disease are displayed.

The inspection period display section R2 is a section in which an inspection period of the patient is displayed and a period of inspection received by the patient is displayed by a horizontal bar graph. The medication period display section R3 is a section in which a medication period or a drip infusion period of the patient is displayed and a period of medication or drip infusion received by the patient is displayed by a horizontal bar graph.

The inspection data display section R4 is a section in which inspection data of a plurality of inspection items obtained in time series are displayed by line graphs. The horizontal axis of the inspection data display section R4 is a time axis while the vertical axis represents numerical values of the inspection data. The inspection data display section R4 is provided with a scroll bar B1 for changing the numerical range of the inspection data to be displayed and a scroll bar B2 for changing the display period of the inspection data.

The image examination information section R5 is a section in which image examination information of the patient is displayed. More specifically, a thumbnail image of an image captured by imaging the patient with a CT system or a MRI system is displayed. When the thumbnail image is selected, for example, by being clicked, the original image of the thumbnail image is displayed on a separate screen and an image interpretation report of the original image is also displayed.

The medication list display section R6 is a section in which types of medicines administered or drip-infused during each medication period displayed in the medication period display section R3 are list-displayed. In addition to the list-display of medicines, a checkbox is displayed for each medicine in the medication list display section R6. Then, the medication period of a medicine whose checkbox has been checked is highlighted in the medication period display section R3. More specifically, for example, the medication period of a medicine checked with the checkbox is color-displayed while the medication period of a medicine not checked with the checkbox is displayed in pale gray or similar color so as to be less noticeable.

The inspection item list display section R7 is a section in which inspection items of the inspection data displayed in the inspection data display section R4 are list-displayed. In addition to the list-display of inspection items, a checkbox is displayed for each inspection item in the inspection item list display section R7. Then, the inspection data of an inspection item whose checkbox has been checked are highlighted in the inspection data display section R4. More specifically, for example, the inspection data of an inspection item checked with the checkbox are color-displayed while the inspection data of an inspection item not checked with the checkbox are displayed in pale gray or similar color so as to be less noticeable.

Now, returning to FIG. 1, the display period specification receiving unit 14 receives a specification of a display period of inspection data to be displayed and outputs the display period information to the medical care information obtaining unit 11. The display period is a display period of inspection data a doctor or a similar person wants to observe with a particular attention and, for example, the date of a display start day and the date of a display end day are inputted and the period from the display start day to the display end day is the display period, and the inspection data during this display period are display target inspection data. Further, a display start month and a display end moth may be specified, instead of dates.

Then, the medical care information obtaining unit 11 described above identifies an inspection item whose inspection data are present in the display period described above from many inspection items of the inspection data stored in an inspection storage server 41, to be described later, of the inspection department system 40 based on the inputted display period information, and obtains inspection data of the inspection item. Then the medical care information obtaining unit 11 outputs the obtained inspection data to the display control unit 13.

Based on the inputted inspection data and the specified display period, the display control unit 13 displays a graph of the inspection data in the inspection data display section R4 described above. The display method of the inspection data in the inspection data display section R4 will be described in detail later.

The diagnosis and treatment department terminal 20 is provided with a display, a keyboard, a mouse, and the like. The display of the diagnosis and treatment department terminal 20 displays the medical care information display screen shown in FIG. 2. The keyboard and the mouse of the diagnosis and treatment department terminal 20 receive an input of identification information of a patient, an input of the aforementioned display period of inspection data, a specifying input of the aforementioned checkbox, and the like.

The electronic medical record system 30 is a system that manages electronic medical records of a plurality of patients. The electronic medical record system 30 is provided with an electronic medical record storage server 31, and the electronic medical record storage server 31 includes electronic medical records of a plurality of patients associated with identification information of the patients. The electronic medical record system 30 obtains identification information of a patient inputted at the diagnosis and treatment department terminal 20 and outputs the electronic medical record information associated with the identification information to the medical care information display control apparatus 10. An electronic medical record includes information, such as basic information of a patient, a type of a medicine administered or drip-injected and a medication period, a history of disorders, and the like.

The inspection department system 40 is a system that manages inspection data of a plurality of patients. The inspection department system 40 is provided with an inspection data storage server 41, and the inspection data storage server 41 includes inspection data of a plurality of inspection items of each patient associated with the identification information of each patient. The inspection department system 40 obtains identification information of a patient inputted at the diagnosis and treatment department terminal 20 and outputs the inspection data associated with the identification information to the medical care information display control apparatus 10.

The image diagnosis system 50 is a system that manages image examination information of a plurality of patients. The image diagnosis system 50 is provided with an image management server 51 and an image interpretation report server 52. The image management server 51 includes a radiation image or a similar image of each patient associated with the identification information of each patient. The image interpretation report server 52 includes an image interpretation report generated by a doctor through observation of a radiation image or a similar image, and the image interpretation report is stored associated with the radiation image or the similar image stored in the image management server 51. The image diagnosis system 50 obtains identification information of a patient inputted at the diagnosis and treatment department terminal 20 and outputs a radiation image and an image interpretation report of the image associated with the identification information to the medical care information display control apparatus 10.

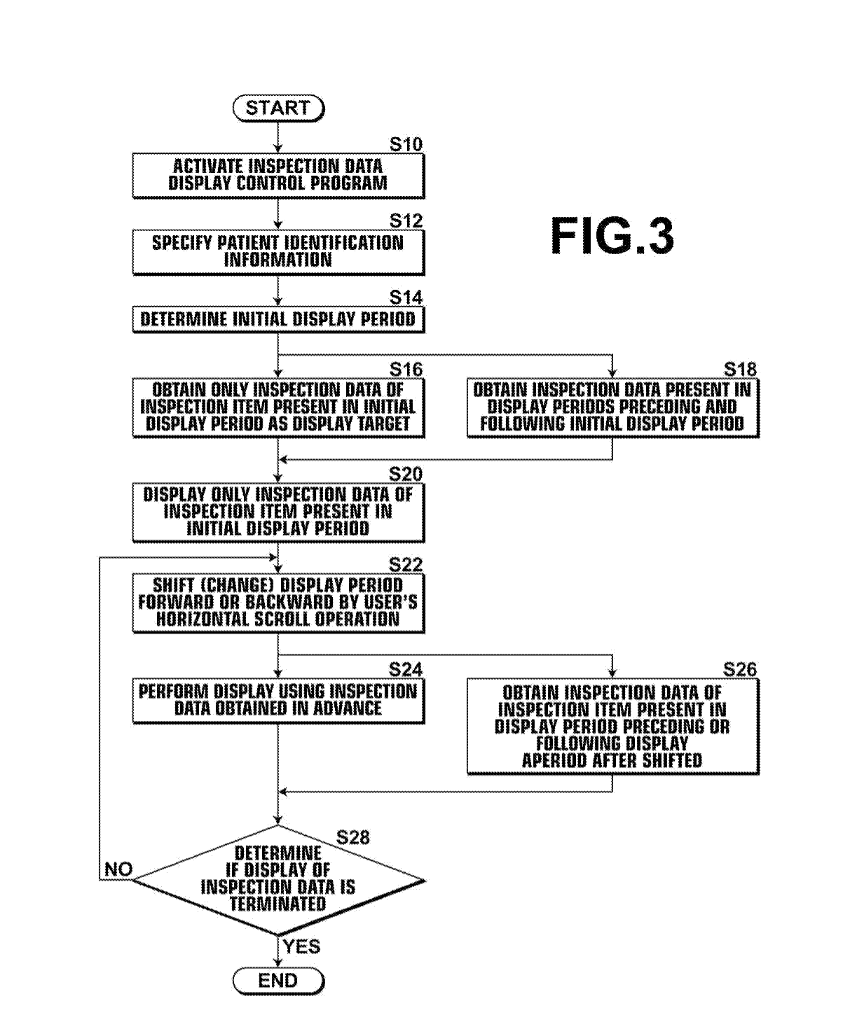

Next, an operation of the medical care information display system 1 of the present embodiment will be described with reference to a flowchart shown in FIG. 3. Since the medical care information display system 1 has a distinguishing feature in the display method of inspection data in the inspection data display section R4 described above, a description will be made, hereinafter, focusing mainly on that point.

First, the inspection data display control program stored in the medical care information control apparatus 10 is activated through an instruction input at the diagnosis and treatment department terminal 20 by a doctor or a similar person (S10).

Next, identification information of a patient is inputted at the diagnosis and treatment department terminal 20 and the identification information of a patient is obtained by the medical care information obtaining unit 11 of the medical care information display control apparatus 10 (S12).

Next, when an initial display period of inspection data to be displayed is inputted at the diagnosis and treatment department terminal 20, information of the initial display period is obtained by the display period specification receiving unit 14, and the display period specification receiving unit 14 outputs the information of the initial display period to the medical care information obtaining unit 11 (S14).

Based on the inputted identification information of a patient, the medical care information obtaining unit 11 will read out inspection data of the patient from the inspection data storage server 41 of the inspection department system 40. But, first, the medical care information obtaining unit 11 identifies an inspection item of inspection data present in the aforementioned initial display period. Then, the medical care information obtaining unit 11 reads out and obtains only the inspection data of the aforementioned identified inspection item present in the aforementioned initial display period among inspection data of multiple inspection items stored in the inspection data storage server 41, as display target inspection data of the aforementioned initial display period (S16).

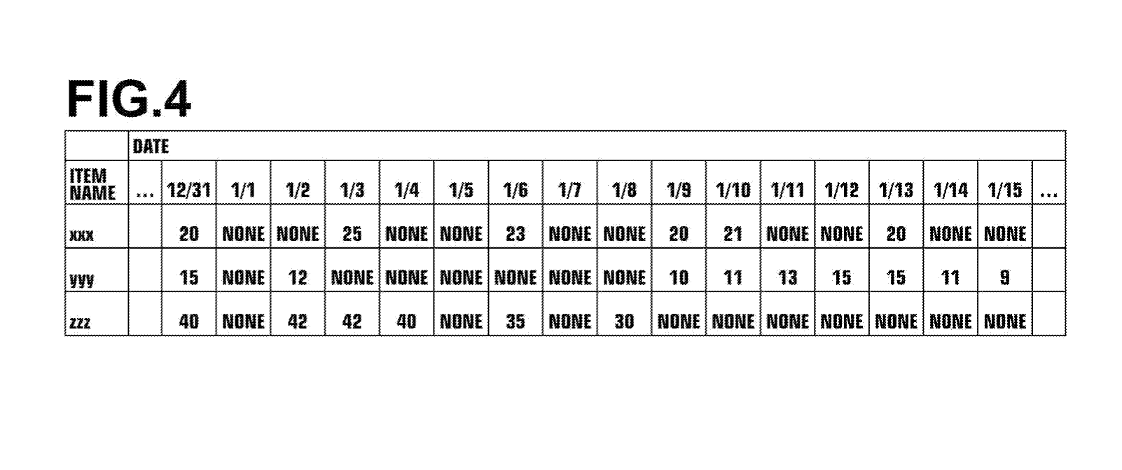

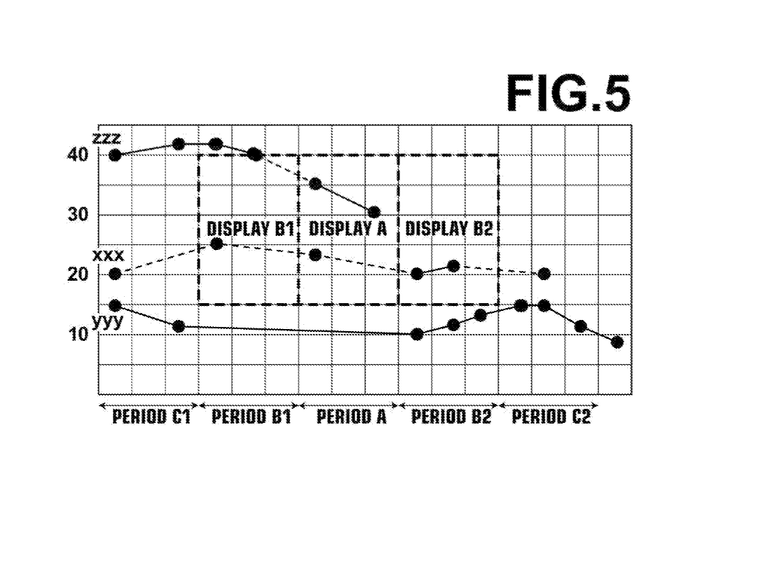

Hereinafter, a description will be made by taking a specific example. Here, it is assumed that inspection data of three inspection items “xxx”, “yyy”, and “zzz”, as shown in FIG. 4, are stored in the inspection data storage server 41 for a patient specified by a doctor. FIG. 5 plots all inspection data of the three items shown in FIG. 4. The horizontal axis represents the date in FIG. 4 and the vertical axis represents numerical values of inspection data.

When a display period A shown in FIG. 5 is specified as the aforementioned initial display period, the medical care information obtaining unit 11 identifies two inspection items “xxx” and “zzz” present in the display period A from the three inspection items and obtains only the inspection data present in the display period A of the two inspection items “xxx” and “zzz” from the inspection data of the three items shown in FIG. 5, as display target inspection data of the display period A.

At this time, the medical care information obtaining unit 11 identifies an inspection item present in the display periods preceding and following the initial display period, and obtains inspection data of the identified inspection item in the display periods preceding and following the initial display period in advance (S18). That is, when the display period A is specified as the initial display period, an inspection item present in the display periods B1, B2 preceding and following the display period A is identified, and the inspection data of the inspection item present in the display periods B1, B2 are obtained in advance. In the example shown in FIG. 5, inspection data of the two inspection items “xxx” and “zzz” present in the display period B1 and inspection data of the inspection item “xxx” present in the display period B2 are obtained in advance.

In the present embodiment, the display periods B1, B2, which are the periods preceding and following the initial display period, are set to the same period of three days as the initial display period, but the period for obtaining inspection data in advance is not necessarily the same as the initial display period A. More specifically, any other period may be set as long as it is longer than a period of a minimum unit of change (shift) of the display period to be described later. Further, an arrangement may be adopted in which the period for obtaining inspection data in advance is changed arbitrarily by the user.

Then, the inspection data of inspection items “xxx” and “zzz” present in the display period A and display periods B1, B2 obtained by the medical care information obtaining unit 11 are temporarily stored in the medical care information storage unit 12.

Next, the display control unit 13 sets the horizontal axis of the inspection data display section R4 shown in FIG. 2 to the display period A, which is the initial display period, obtains inspection data of the inspection items “xxx” and “zzz” in the display period A stored in the medical care information storage unit 12, and displays only the obtained inspection data in the inspection data display section R4 (S20). That is, graphs of the inspection data in the display period A shown in FIG. 5 are displayed in the inspection data display section R4.

At this time, inspection data present in the display period A are displayed connected with each other while the inspection data present in the display period A and inspection data present in the display periods B1, B2 are not connected in the present embodiment. That is, the portions indicated by the dotted lines in FIG. 5 are not connected. The omission of the connections in the manner described above may provide a more easy-to-view display.

Next, when the display period to be displayed is changed from the display period A to a display period preceding or following the display period A by the user, such as a doctor or a similar person, information of the display period after the change is obtained by the display period specification receiving unit 14 (S22). Note that the display period is changed with the time axis scroll bar B2 of the inspection data display section R4, but not limited to this and a display period after the change may be set and inputted using the keyboard of the diagnosis and treatment department terminal 20 or the like.

Then, the information of the display period after the change obtained by the display period specification receiving unit 14 at this time is outputted to the display control unit 13. When the information of the display period after the change is received, the display control unit 13 displays inspection data present in the display period after the change using the inspection data obtained and stored in the medical care information storage unit 12 in advance in S18 (S24).

More specifically, for example, when the display period A is changed to the display period B1 shown in FIG. 5, the display control unit 13 sets the horizontal axis of the inspection data display section R4 to the display period B1 and displays inspection data of the display period B1 in the inspection data display section R4 using the inspection data of the display period B1 stored in the medical care information storage unit 12 in advance. Here also, inspection data present in the display period B1 are connected with each other, but the inspection data present in the display period B1 and inspection data present in the display periods preceding and following the display period B1 are not connected. The portions indicated by the dotted lines in FIG. 5 are not connected.

Further, for example, when the display period A is changed to the display period B2 shown in FIG. 5, the display control unit 13 sets the horizontal axis of the inspection data display section R4 to the display period B2 and displays inspection data of the display period B2 in the inspection data display section R4 using the inspection data of the display period B2 stored in the medical care information storage unit 12 in advance. Here also, inspection data present in the display period B2 are connected with each other, but the inspection data present in the display period B2 and inspection data present in the display periods preceding and following the display period B2 are not connected. The portions indicated by the dotted lines in FIG. 5 are not connected.

In this way, when the display period A, which is the initial display period, is specified, inspection data of an inspection item present in the display periods B1, B2 preceding and following the display period A are obtained in advance and, when the display period A is changed to a display period preceding or following the display period A, inspection data present in the display period after the change are displayed using the inspection data obtained in advance. This allows inspection data to be displayed faster than in the case in which inspection data are read out from the inspection data storage server 41 after the display period is changed.

In the meantime, when the display period to be displayed is changed from the display period A to the display period preceding or following the display period A as described above, information of the display period after the change obtained by the display period specification receiving unit 14 is also outputted to the medical care information obtaining unit 11.

When the information of the display period after the change is obtained, the medical care information obtaining unit 11 identifies an inspection item present in the display period preceding or following the display period after the change and obtains inspection data of the identified inspection item in the display period preceding or following the display period after the change in advance (S26). More specifically, when the display period A is changed to the display period B1, the medical care information obtaining unit 11 identifies an inspection item present in a display period C1 from which inspection data have not been obtained yet, obtains inspection data of the identified inspection item in the display period C1 by reading out from the inspection data storage server 41, and stores the obtained inspection data in the medical care information storage unit 12.

Further, when the display period A is changed to the display period B2, the medical care information obtaining unit 11 identifies an inspection item present in a display period C2 from which inspection data have not been obtained yet, obtains inspection data of the identified inspection item in the display period C2 by reading out from the inspection data storage server 41, and stores the obtained inspection data in the medical care information storage unit 12.

Then, when the display period is changed by the user again from the display period B1 or B2 to a display period preceding or following the display period B1 or B2 (S22), without terminating the display of inspection data (S28, NO), the display control unit 13 sets the horizontal axis of the inspection data display section R4 to the display period after the change, and displays inspection data of the changed display period in the inspection data display section R4 using the inspection data of the display period C1, the display period C2, or the display period A stored in the medical care information storage unit 12 in advance (S24). At this time, the medical care information obtaining unit 11 identifies an inspection item present in a display period preceding or following the display period after the change, as in the manner described above, and obtains inspection data of the identified inspection item which have not been obtained yet by reading out from the inspection data storage server 41, and stores the obtained inspection data in the medical care information storage unit 12.

Then, the processing of S22 to S26 is repeated until an instruction to terminate the display of inspection data is inputted (S28, YES).

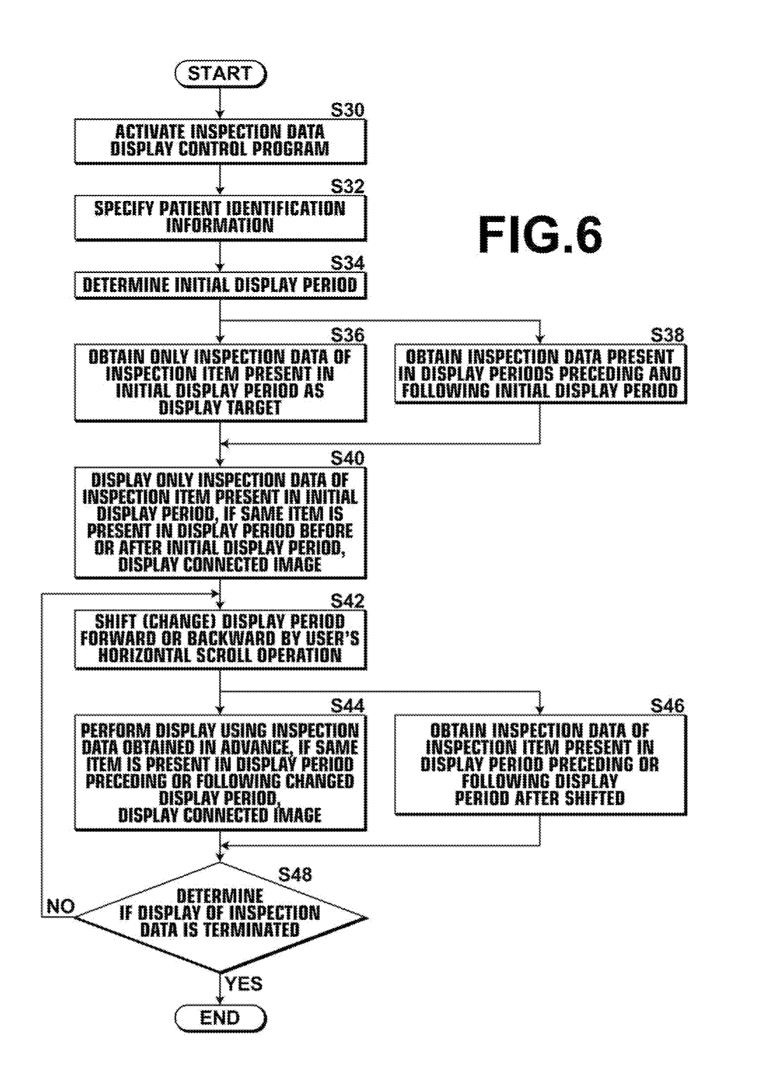

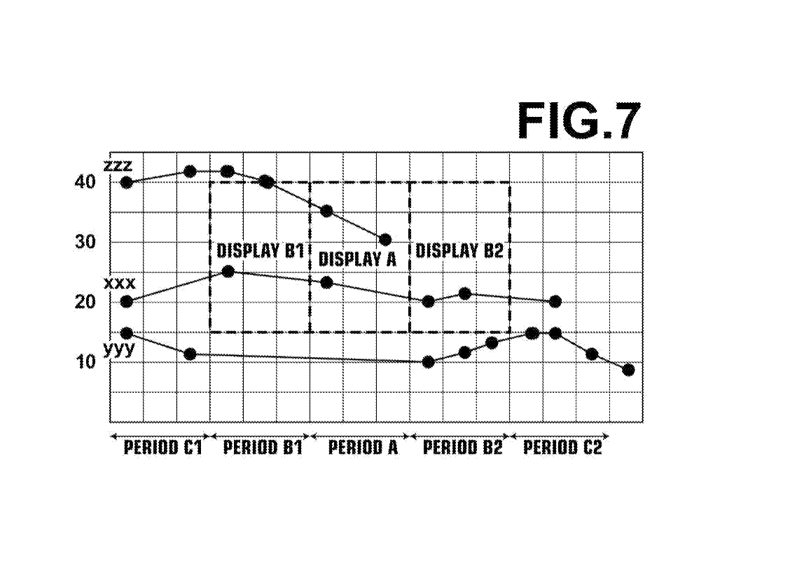

Next, a medical care information display system 1 that uses a second embodiment of the present disclosure will be described. The medical care information display system 1 of the second embodiment differs from the medical care information display system 1 of the foregoing first embodiment in that, while in the first embodiment, when displaying inspection data of a given display period in the inspection data display section R4, only the inspection data present in the display period are connected with each other and the inspection data present in the display period and inspection data present in the display periods preceding and following the given display period are not connected, in the second embodiment, inspection data present in a given display period and inspection data present in the display periods preceding and following the given display period are connected and displayed. As the configuration and operation of the second embodiment are identical to those of the first embodiment other than this, a description will be made with reference to a flowchart of FIG. 6, and FIG. 7, focusing mainly on the different point.

The processing of S30 to S38 in the flowchart of FIG. 6 is identical to the processing of S10 to S18 in the flowchart of FIG. 3.

Then, in the second embodiment, when displaying inspection data of an inspection item present in the initial display period A in the inspection data display section R4, if inspection data of the same inspection item as that present in the display period A are present in a display period B1 or in a display period B2, which are the display periods preceding and following the display period A, the display control unit 13 displays the inspection data present in the display period A by connecting the data with the inspection data of the same inspection item in the display period B1 or in the display period B2, as shown in FIG. 7 (S40).

Thereafter, if a display period is changed (S42), inspection data of the display period after the change are displayed in the inspection data display section R4 using inspection data obtained and stored in advance in the medical care information storage unit 12, as in the first embodiment. In this case also, if inspection data of the same inspection item as that present in the display period after the change are present in the display period preceding or following the display period after the change, the display control unit 13 displays the inspection data present in the display period after the change by connecting the data with the inspection data present in the display period preceding or following the display period after the change (S44).

More specifically, if the display period after the change is the display period B1 and inspection data of the same inspection item as that in the display period B1 are present in the display period C1 or in the display period A, which are display periods preceding and following the display period B1, the display control unit 13 displays the inspection data present in the display period B1 by connecting the data with the inspection data of the same inspection item present in the display period C1 or in the display period A, as shown in FIG. 7. If the display period after the change is the display period B2 and inspection data of the same inspection item as that in the display period B2 are present in the display period C2 or in the display period A, which are display periods preceding and following the display period B2, the display control unit 13 displays the inspection data present in the display period B2 by connecting the data with the inspection data of the same inspection item present in the display period C2 or in the display period A, as shown in FIG. 7.

The description for S46 to S48 are omitted here as they are identical to S26 to S28 in the flowchart of FIG. 3.

According to the medical care information display system 1 of the second embodiment, if the same display item as that of the display period currently displayed is present in the display period preceding or following the display period currently displayed, the inspection data of the inspection item in the display period currently displayed are displayed by connecting the inspection data with the inspection data of the same inspection item in the display period preceding or following the display period currently displayed. This allows the user to be notified that inspection data of the same inspection item are present in the currently not displayed display period preceding or following the currently displayed period by displaying the connection.

Here, when the display period is changed, as in the first and the second embodiments, if the numerical value range of the inspection data present in the display period after the change is different largely from the numerical value range of the inspection data present in the display period before the change, there may be a case in which nothing is displayed on the screen immediately after changing the display period. Then, in such a case, it is necessary to search for inspection data by, for example, scrolling and changing the numerical value range of the axis representing numerical values for displaying the inspection data of the display period after the change on the screen. This takes time to refer to the inspection data, which may lead to a decrease in efficiency of medical care after all.

Next, a medical care information display system 1 of a third embodiment that solves the foregoing problem will be described.

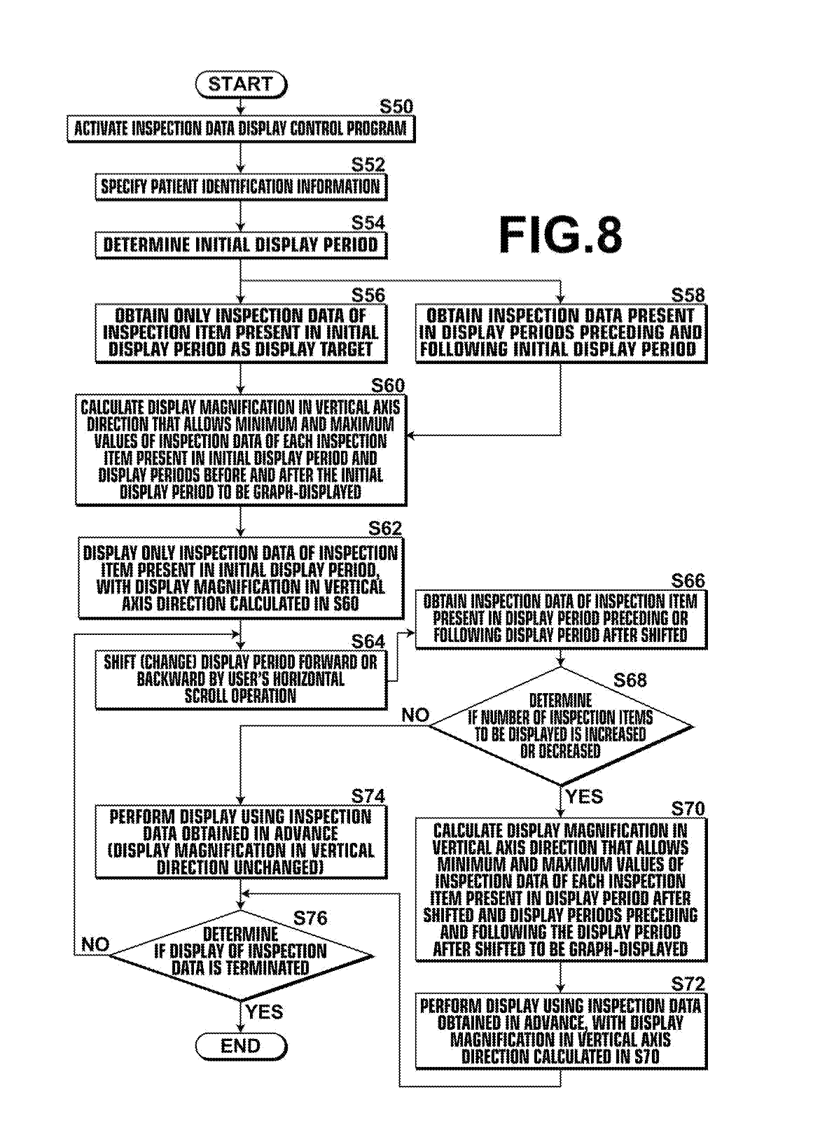

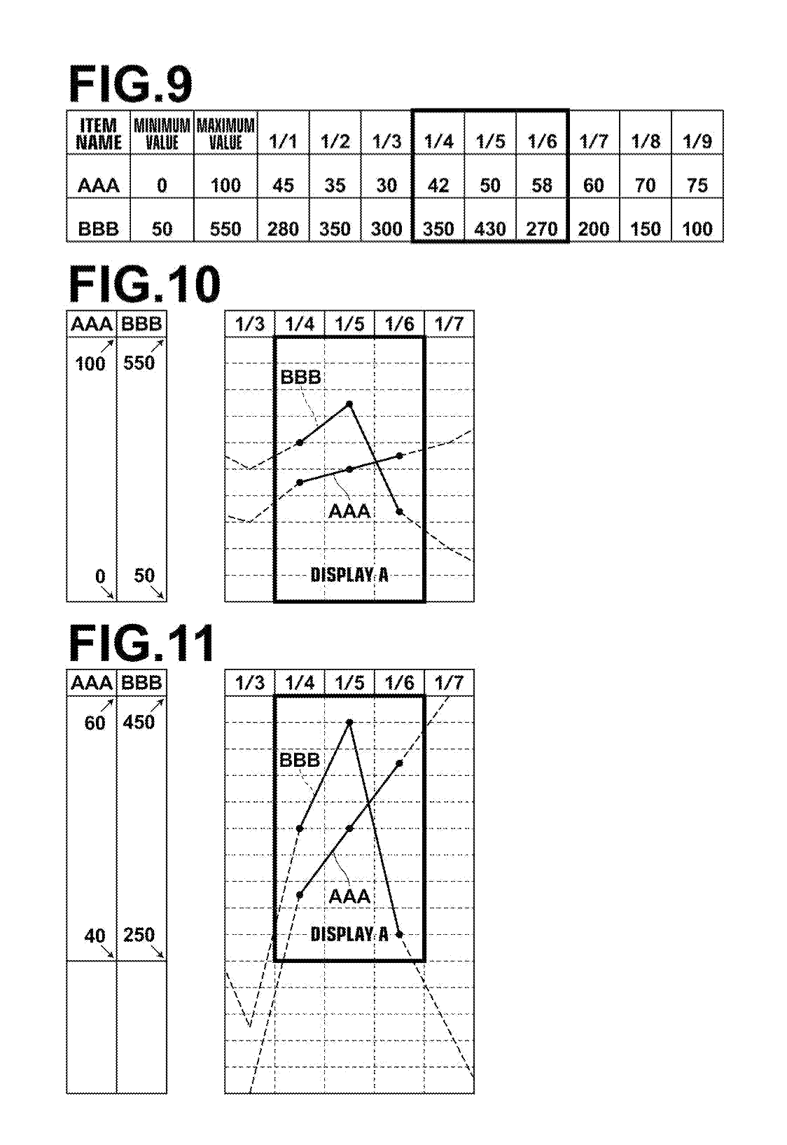

When displaying inspection data by changing the display period, as in the aforementioned first and second embodiments, the medical care information display system 1 of the third embodiment changes the display magnification of a vertical axis direction of a graph of inspection data in the display area (inspection data display section R4) to prevent the inspection data from stretching out of the display area. Since the configuration and operation of the third embodiment are identical to those of the first and second embodiments other than this, a description will be made with reference to a flowchart of FIG. 8, and FIGS. 9, 10, focusing mainly on the different point.

The processing of S50 to S58 in the flowchart of FIG. 8 is identical to the processing of S10 to S18 in the flowchart of FIG. 3. Note that it is assumed here that inspection data of two inspection items “AAA” and “BBB”, as shown in a table of FIG. 9, are stored in the inspection data storage server 41 for a patient specified by a doctor. It is also assumed that the period of ¼ to ⅙ is specified as the initial display period.

After inspection data present in the initial display period and inspection data present in the display periods preceding and following the initial display period are obtained, an inspection item in the initial display period and in the display periods preceding and following the initial display period is identified, and maximum and minimum values of the inspection data of the identified inspection item are obtained in the third embodiment. More specifically, as the inspection items in the initial display period (1/4 to 1/6) and in the display periods preceding and following the initial display period are “AAA” and “BBB” in the example shown in the table of FIG. 9, these inspection items are identified, and maximum and minimum values of these inspection items are obtained. If the inspection item differs between the initial display period and the display periods preceding and following the initial display period, all the inspection items in the initial display period and in the display periods preceding and following the initial display period are identified, and maximum and minimum values of all the identified inspection items are obtained.

For the maximum and minimum values obtained here are those of inspection data that include not only the inspection data present in the initial display period and in the display periods preceding and following the initial display period but also the inspection data stored in the inspection data storage server 41. It is assumed here that values of 100 and 0 are obtained as the maximum value and the minimum value of the inspection item “AAA” respectively, while values of 550 and 50 are obtained as the maximum value and the minimum value of the inspection item “BBB” respectively, as shown in FIG. 9.

The maximum value and the minimum value obtained for each of the inspection items “AAA” and “BBB” are outputted to the display control unit 13.

Based on the inputted maximum value and the minimum value of each inspection item, the display control unit 13 calculates a display magnification in a vertical axis direction that allows all these values to be displayed in the graph display area (inspection data display section R4) (S60). That is, if the maximum and minimum value range of each inspection item is as shown in the table of FIG. 9, a display magnification that allows the maximum value 100 and the minimum value 0 of the inspection item “AAA” to be displayed in the graph display area is calculated for the vertical axis direction of the inspection item “AAA” and a display magnification that allows the maximum value 550 and the minimum value 5 of the inspection item “BBB” to be displayed in the graph display area is calculated for the vertical axis direction of the inspection item “BBB”, and the display control unit 13 compresses the vertical axis of the inspection item “AAA” and the vertical axis of the inspection item “BBB” based on the display magnifications. The display magnification as used herein is determined by the range of inspection data values allocated to the length of the vertical axis of the graph display area and, for example, in a case where the numerical range of inspection data is 0 to 50, the display magnification is double of that of a case where the numerical range is 0 to 100.

Then, the display control unit 13 displays only the inspection data of an inspection item in the initial display period A obtained in the step S56 in the inspection data display section R4, and a graph of inspection data of each inspection item here is compressed based on the aforementioned display magnification (S62). That is, as shown in FIG. 10, the graph of the inspection data is compressed such that the maximum value 100 and the minimum value 0 fall within the display area indicated by the thick frame and displayed for the inspection item “AAA”, while the graph of the inspection data is compressed such that the maximum value 550 and the minimum value 50 fall within the display area indicated by the thick frame and displayed for the inspection item “BBB”.

FIG. 11 shows an example in which the vertical axis is set such that only the inspection data present in the initial display period fall within the display area. In this case, if the display period is changed, inspection data not displayed in the display area may exist.

In contrast, if the vertical axis is compressed based on a maximum value and a minimum value of inspection data of each inspection item as described above, even when the display period is changed, inspection data of the display period after the change may be prevented from stretching out from the graph display area. When displaying inspection data present in the initial display period A, FIG. 10 shows that the inspection data present in the initial display period A are not connected to the inspection data present in the display periods preceding and following the initial display period A, but they may be connected, as in the second embodiment.

Next, when the initial display period is changed to a display period preceding or following the initial display period by the user (S64), the medical care information obtaining unit 11 identifies an inspection item present in a display period preceding or following the display period after the change and obtains inspection data of the identified inspection item present in the display period preceding or following the display period after the change, as in the first and the second embodiments (S66).

At this time, the display control unit 13 identifies a display item in the display period after the change and a display item in the display periods preceding and following the display period after the change and obtains maximum and minimum values of inspection data of the identified inspection items.

When the display period is changed in this way, if the number of inspection items in the display period after the change and in the display periods preceding and following the display period after the change does not change from the number of inspection items before the change (S68, NO), the display control unit 13 displays inspection data of the display period after the change obtained and stored in the medical care information storage unit 12 in advance in the display area without making a change in the vertical axis of the display area (S74).

On the other hand, when the display period is changed, if the number of inspection items in the display period after the change and in the display periods preceding and following the display period after the change increases or decreases from the number of inspection items before the change (S68, YES), inspection items included in the display period after the change and in the display periods preceding and following the display period after the change are identified, and maximum and minimum values are obtained for each of all the identified inspection items. Then, the maximum and minimum values obtained for each inspection item are outputted to the display control unit 13.

Based on the inputted maximum value and the minimum value of each inspection item, the display control unit 13 calculates a display magnification in the vertical axis direction that allows all these values to be displayed in the graph display area (inspection data display section R4) (S70).

Then, the display control unit 13 compresses the vertical axis of the graph display area based on the display magnification calculated in the manner described above and displays the inspection data obtained and stored in the medical care information storage unit 12 in advance in the display area (S72).

Then, the processing of S64 to S74 is repeated until an instruction to terminate the display of inspection data is inputted (S76, YES).

As described above, when the display period is changed, if inspection items included in the display period after the change and in the display periods preceding and following the display period after the change are identified and the vertical axis of the display area is compressed based on a display magnification calculated from maximum and minimum values of the identified inspection items, inspection data are always prevented from stretching out from the display area even when the number of inspection items increases or decreases after the display period is changed.

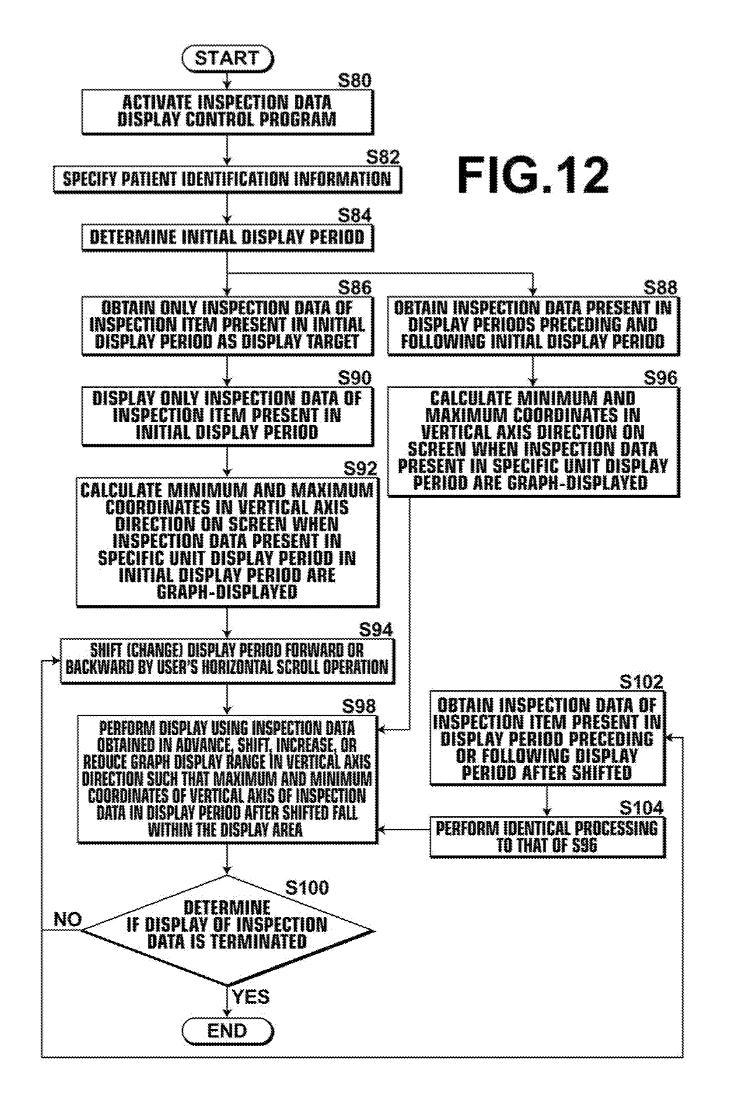

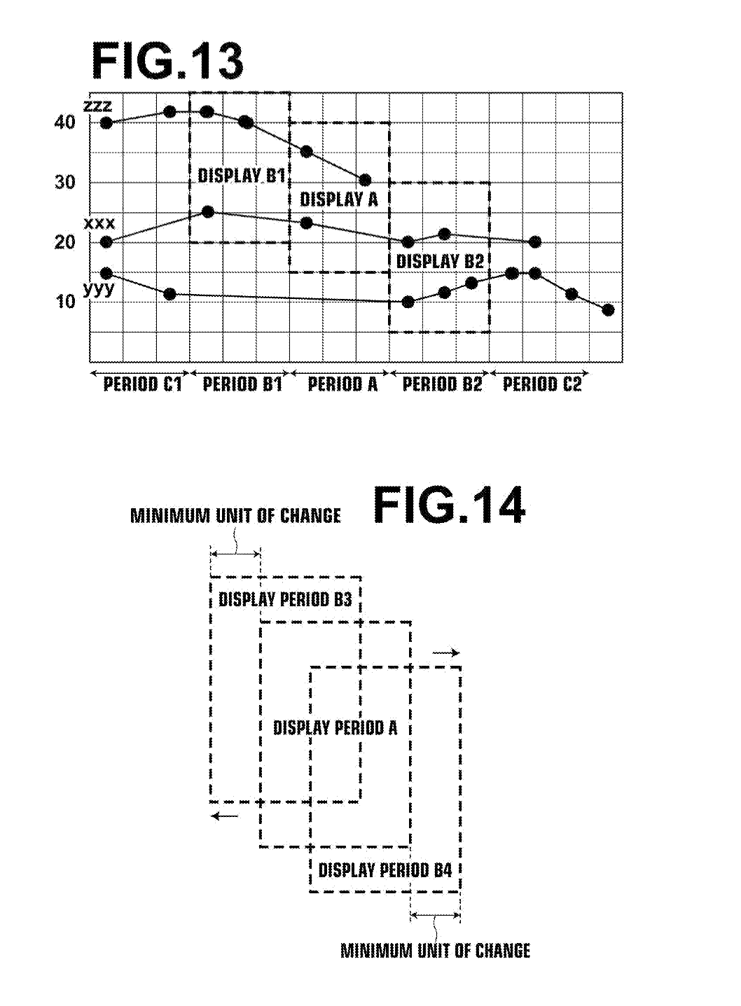

Next, a medical care information display system 1 that uses a fourth embodiment of the present disclosure will be described. In the medical care information display system 1 of the third embodiment described above, the vertical axis is compressed such that inspection data are always prevented from stretching out from the display area even when the display period is changed. The medical care information display system 1 of the fourth embodiment does not compress the vertical axis in the manner described above but, instead, obtains, with inspection data present in the display period after the change, maximum and minimum coordinates of the obtained inspection data on an as-needed basis, and automatically changes the vertical axis of the display area according to the maximum and minimum coordinates, whereby the inspection data of the display period are prevented from stretching out from the display area. That is, the numerical range of the vertical axis is automatically scrolled according to inspection data present in the display period after the change. Hereinafter, a description will be made in detail with reference to a flowchart of FIG. 12, and FIG. 13.

The processing of S80 to S90 in the flowchart of FIG. 12 is identical to the processing of S10 to S20 in the flowchart of FIG. 3. Here, it is assumed that inspection data of three inspection items “xxx”, “yyy”, and “zzz”, as shown in FIG. 4, are stored in the inspection data storage server 41 for a patient specified again by a doctor. FIG. 13 plots all inspection data of the three items shown in FIG. 4.

After obtaining inspection data present in the display periods preceding and following the initial display period, the fourth embodiment calculates maximum and minimum coordinates in the vertical axis direction on the screen (display area) when inspection data present in a specific unit display period are graph-displayed with respect to the inspection data present in the display periods preceding and following the initial display period (S96). Note that the unit display period described above is preferable, for example, to be a minimum unit of change (shift) in horizontal axis, but not limited to this and may be any period as long as it is longer than the minimum unit and shorter than or equal to the display periods preceding and following the initial display period.

More specifically, if the initial display period is display period A shown in FIG. 13, minimum and maximum coordinates in the vertical axis direction on the screen when inspection data present in the unit display period are graph-displayed are calculated with respect to the inspection data present in the display period B1. In addition, minimum and maximum coordinates in the vertical axis direction in the unit display period is also calculated in the same manner as described above for the inspection data present in the display period B2.

In the meantime, minimum and maximum coordinates in the vertical axis direction in the specific unit display period is also calculated with respect to the inspection data present in the initial display period (S92).

When the initial display period is changed to the display period preceding or following the initial display period (S94), the display control unit 13 displays the inspection data present in the display period preceding or following the initial display period in the inspection data display section R4. At this time, maximum and minimum coordinates in the vertical axis direction calculated for the inspection data present in the display period after the change are obtained, and the graph display range is automatically shifted in a vertical axis direction such that the maximum and minimum coordinates fall within the display area of the inspection data display section R4, and further the display range is increased or reduced, as required (S98).

More specifically, as shown in FIG. 13, when the initial display period A is changed to the display period B1, the vertical axis of the display area of the inspection data display section R4 automatically scrolls and the dotted range of the display period B1 shown in FIG. 13 is displayed in the inspection data display section R4. Also, when the initial display period A is changed to the display period B2, the vertical axis of the display area of the inspection data display section R4 automatically scrolls and the dotted range of the display period B2 shown in FIG. 13 is displayed in the inspection data display section R4.

FIG. 13 shows an example in which the display period A is changed to the range of display period B1 or B2, but the display period change is not limited to these and the display period may be changed sequentially with the minimum unit of change in horizontal axis described above. Then, as illustrated in FIG. 14, each time a change is made with the minimum unit, maximum and minimum coordinates in a vertical axis direction calculated for the inspection data present in the display period B3 or B4, and the graph display range is automatically shifted such that the maximum and minimum coordinates fall within the display area of the inspection data display section R4 or the display range is increased or reduced, as required.

Then, if an instruction to terminate the display of inspection data is not issued (S100, NO), inspection data of an inspection item in the display period preceding or following the display period after the change are obtained and the inspection data are stored in the medical care information storage unit 12 (S102).

Then, processing identical to that of S96 is performed and minimum and maximum coordinates in the vertical axis direction on the screen when inspection data present in the specific unit display period are graph-displayed are calculated with respect to the inspection data present in the display periods preceding and following the display period after the change (S104).

Then, if the display period is changed again by the user (S94), the processing of S98 is repeated.

The processing of S94 to S104 is repeated until an instruction to terminate the display of inspection data is inputted (S100, YES).

According to the medical care information display system 1 of the fourth embodiment, the vertical axis is made to scroll automatically, along with the change of the display period, such that the numerical range in which the inspection data present in the display period after the change are present corresponds to the display area of the inspection data display section R4. This eliminates the need for the user to search for the range in which inspection data are present, and the user may refer to the inspection data immediately.

What is claimed is:

1. An inspection data display control apparatus for displaying inspection data of a plurality of inspection items obtained in time series, the apparatus comprising:
a display period specification receiving unit that receives a specification of a display period of inspection data to be displayed;
an inspection data obtaining unit that, when the specification of the display period is received, identifies an inspection item whose inspection data are present in the display period from the plurality of inspection items and obtains only the inspection data of the identified inspection item, as display target inspection data present in the display period; and
a display control unit that displays only the inspection data of the inspection item present in the display period.

2. The inspection data display control apparatus of claim 1, wherein:
the display period specification receiving unit receives a specification of a display period changed from the display period;
when the specification of the display period before the change is received, the inspection data obtaining unit identifies an inspection item present in display periods preceding and following the display period before the change, and obtains inspection data of the identified inspection item in the display periods preceding and following the display period before the change in advance before the change in the display period; and
when the change in the display period is received, the display control unit displays inspection data present in the display period after the change using the inspection data obtained in advance.

3. The inspection data display control apparatus of claim 1, wherein:
when the specification of the display period is received, the inspection data obtaining unit identifies an inspection item present in display periods preceding and following the received display period, and obtains inspection data of the identified inspection item in the display periods preceding and following the received display period in advance; and
when displaying the inspection data of the inspection item present in the display period, the display control unit, if inspection data of the same inspection item as that present in the display period is present in the display periods preceding and following the display period, displays the inspection data present in the display period by connecting the inspection data present in the display period with the inspection data of the same inspection item present in the display periods preceding and following the display period.

4. The inspection data display control apparatus of claim 2, wherein when displaying the inspection data present in the display period before the change, the display control unit identifies an inspection item present in the display periods preceding and following the display period before the change, and automatically changes a numerical value range of an axis representing numerical values such that maximum and minimum values of each identified inspection item are all displayed in a display area.

5. The inspection data display control apparatus of claim 2, wherein when displaying the inspection data present in the display period after the change, the display control unit automatically shifts a numerical value range of an axis representing numerical values such that both maximum and minimum values of the inspection data of the inspection item present in the display period after the change are included in a display area.

6. The inspection data display control apparatus of claim 5, wherein the display control unit shifts the numerical value range by scrolling the axis representing numerical values.

7. The inspection data display control apparatus of claim 5, wherein the display control unit automatically shifts the numerical value range of the axis representing numerical values each time a period of a minimum unit of change of the display period is changed.

8. The inspection data display control apparatus of claim 6, wherein the display control unit automatically shifts the numerical value range of the axis representing numerical values each time a period of a minimum unit of change of the display period is changed.

9. The inspection data display control apparatus of claim 2, wherein the display control unit displays a scroll bar for instructing a change in the display period.

10. The inspection data display control apparatus of claim 1, wherein the inspection data obtaining unit obtains the inspection data via a network.

11. An inspection data display control method for displaying inspection data of a plurality of inspection items obtained in time series, the method comprising the steps of:
receiving a specification of a display period of inspection data to be displayed;
identifying, when the specification of the display period is received, an inspection item whose inspection data are present in the display period from the plurality of inspection items and obtaining only the inspection data of the identified inspection item, as display target inspection data present in the display period; and
displaying only the inspection data of the inspection item present in the display period.

12. A non-transitory computer-readable recording medium containing an inspection data display control program for displaying inspection data of a plurality of inspection items obtained in time series, the program causing a computer to function as:
a display period specification receiving unit that receives a specification of a display period of inspection data to be displayed;
an inspection data obtaining unit that, when the specification of the display period is received, identifies an inspection item whose inspection data are present in the display period from the plurality of inspection items and obtains only the inspection data of the identified inspection item, as display target inspection data present in the display period; and
a display control unit that displays only the inspection data of the inspection item present in the display period.
